(19) United States
(12) Patent Application Publication
(10) Pub. No.: US 2024/0306930 A1
(43) Pub. Date: Sep. 19, 2024

(54) CATHETER SPLINES AS LOCATION SENSORS

(71) Applicant: Biosense Webster (Israel) Ltd., Yokneam (IL)

(72) Inventors: Meir BAR-TAL, Haifa (IL); Avram Dan MONTAG, Haifa (IL)

(73) Assignee: Biosense Webster (Israel) Ltd., Yokneam (IL)

(21) Appl. No.: 18/676,943

(22) Filed: May 29, 2024

Related U.S. Application Data
(63) Continuation of application No. 15/971,966, filed on May 4, 2018, now Pat. No. 12,029,545.
(60) Provisional application No. 62/512,263, filed on May 30, 2017.

(51) Int. Cl. A61B 5/06; A61B 5/00; A61B 5/01; A61B 5/053; A61B 5/287; A61B 5/341; A61B 17/00; A61B 18/00; A61B 18/14; A61B 34/20; A61B 90/00

(52) U.S. Cl. CPC A61B 5/062; A61B 5/287; A61B 5/341; A61B 5/6853; A61B 5/6858; A61B 5/6859; A61B 17/00; A61B 18/1492; A61B 34/20; A61B 90/39; A61B 5/015; A61B 5/053; A61B 5/064; A61B 2017/00053; A61B 2018/0022; A61B 2018/00267; A61B 2018/00351; A61B 2018/00577; A61B 2034/2051; A61B 2090/3954; A61B 2560/0223

(57) ABSTRACT

A medical probe includes a flexible spline assembly coupled to a distal end of an elongated shaft and including spline conductors. The spline conductors are electrically coupled to each other at a common distal termination, and each spline conductor extends proximally in an arcuate shape. The medical probe includes elongated conductors extending through the elongated shaft and being electrically coupled to a respective spline conductor. A computational system is configured to provide an output to a plurality of radiators to thereby generate alternating magnetic fields, measure electrical signals across pairs of spline conductors induced by the alternating magnetic fiels, and calculate a position and an orientation of an ellipsoid volume encompassed by the spline conductors based on the electrical signals.

### CROSS-REFERENCE TO RELATED APPLICATIONS

This application is a continuation of prior filed U.S. patent application Ser. No. 15/971,966, filed May 4, 2018 (Attorney Docket No.: BIO5766USNP—253757.000386), which claims the benefit of U.S. Provisional Patent Application No. 62/512,263, filed May 30, 2017 (Attorney Docket No.: BIO5766USPSP), the entire contents of each of which is hereby incorporated by reference as if set forth in full herein.

### FIELD OF THE INVENTION

The present invention relates generally to catheter navigation, and particularly to methods and systems for locating the position and orientation of a catheter.

### BACKGROUND OF THE INVENTION

There is a growing competitive drive in the electrophysiology market to develop basket or balloon type, multi-electrode catheters to provide more detailed electrograms, which result in more accurate maps. The usefulness of the information from such a catheter is greatly improved if location information is also available.

U.S. Pat. No. 6,748,255 to Fuimaono, et al., describes a basket catheter that is stated to be useful for mapping the heart. The catheter comprises an elongated catheter body having proximal and distal ends and at least one lumen therethrough, and a basket-shaped electrode assembly is mounted at the distal end of the catheter body.

U.S. Pat. No. 7,155,270 to Solis, et al., describes a catheter that is stated to be useful for simultaneously mapping multiple points within the heart. The catheter includes a mapping assembly including a plurality of flexible spines, each having a free distal end, and the spines are supported by a support structure that permits the spines to be arranged relative to one another.

U.S. Pat. No. 6,529,756 to Phan, et al., describes a probe that may be used to create circumferential lesions in body tissue and that may also be used to perform mapping functions. The probe includes a collapsible/expandable structure that supports electrodes or other operative elements against the body tissue.

U.S. Pat. No. 6,893,439 to Fleischman, and U.S. Pat. No. 6,939,349 to Fleischman, et al., describe an electrode support structure comprising a guide body having at its distal end a flexible spline leg. The spline leg is flexed to define an arcuate shape to facilitate intimate contact against tissue, and an electrode element is carried by the spline leg for movement along its axis.

U.S. Pat. No. 8,346,339 to Kordis, et al., describes a basket style cardiac mapping catheter having a flexible electrode assembly for detection of cardiac rhythm disorders. The catheter includes a plurality of flexible splines having proximal portions, distal portions and medial portions therein between, and there is an anchor for securably affixing the proximal portions of the splines.

U.S. Pat. No. 8,560,086 to Just, et al., describes a family of catheter electrode assemblies that includes a flexible circuit having a plurality of electrical traces and a substrate, a ring electrode surrounding the flexible circuit and electrically coupled with at least one of the plurality of electrical traces, and an outer covering extending over at least a portion of the electrode.

U.S. Pat. No. 8,644,902 to Kordis, et al., describes a method for detection of cardiac rhythm disorders using a basket style cardiac mapping catheter. The method includes providing a basket assembly including a plurality of flexible splines for guiding a plurality of exposed electrodes, and the electrodes are substantially flat electrodes that are substantially unidirectionally oriented towards a direction outside of the basket.

U.S. Patent Application 2015/0208942 to Bar-Tal, et al., describes how catheterization of the heart may be carried out by inserting a probe having electrodes into a heart of a living subject. The probe may be a basket catheter having multiple ribs, each rib having multiple electrodes.

U.S. Patent Application 2015/0366508 to Chou, et al., describes an expandable catheter assembly with flexible printed circuit board electrical pathways. The expandable assembly can comprise a plurality of splines forming a basket array or basket catheter.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

### SUMMARY OF THE DISCLOSURE

An embodiment of the present invention provides an apparatus, including:
a probe having a proximal end and a distal end, the probe being configured to be inserted into an organ of a human patient and defining a probe axis;
at least two conductors positioned on the probe at the distal end;
at least two flexible conductive splines, each conductive spline having a first termination and a second termination, the first terminations being electrically connected together at a region on the probe axis beyond the distal end, each second termination being electrically connected to a respective one of the conductors, the splines being configured to bend into respective arcuate forms that encompass a volume; and
a processor configured to receive voltages induced on the splines via the conductors and to calculate a position and orientation of the volume in response to the received voltages.

In a disclosed embodiment the splines are attached to flexible material forming a balloon catheter. Alternatively, the probe and the splines form a basket catheter.

In a further disclosed embodiment the processor is configured to calculate an ellipticity of the volume in response to the received voltages.

In a yet further disclosed embodiment the apparatus includes at least one magnetic field radiator which generates an alternating magnetic field traversing the volume encompassed by the splines so as to generate the received voltages.

In an alternative embodiment the at least two flexible conductive splines include an even number of splines, and the processor is configured to calculate respective centers and orientations of opposing pairs of splines, and to derive the position and orientation of the volume from the respective centers and orientations.

In a further alternative embodiment the at least two conductors and the at least two flexible conductive splines are distributed symmetrically about the probe axis.

In a yet further alternative embodiment the at least two conductors and the at least two flexible splines are equal in number.

The at least two flexible conductive splines may consist of n splines, where n is an integer greater than or equal to three, and the processor is configured to receive a subset of

$$\\ ( \begin{matrix}n \\ 2\end{matrix} )$$

voltages induced in respective

$$\\ ( \begin{matrix}n \\ 2\end{matrix} )$$

different pairs of splines, and to calculate the position and orientation of the volume in response to the received subset.

Alternatively, the at least two flexible conductive splines may consist of n splines, where n is an integer greater than or equal to two, and the processor is configured to receive

$$\\ ( \begin{matrix}n \\ 2\end{matrix} )$$

voltages induced in respective

$$\\ ( \begin{matrix}n \\ 2\end{matrix} )$$

different pairs of splines, and to calculate the position and orientation of the volume in response to the received

$$\\ ( \begin{matrix}n \\ 2\end{matrix} )$$

voltages.

The processor may be configured to calculate a magnitude of the volume in response to the received voltages. The splines may be attached to flexible material forming a balloon catheter, and the apparatus may further comprise a screen, and the processor may be configured to present on the screen a virtual representation of the balloon catheter in response to the position, the orientation, and the magnitude of the volume.

There is further provided, according to an embodiment of the present invention, a method, including:
configuring a probe, having a proximal end and a distal end, to be inserted into an organ of a human patient, the probe defining a probe axis;
positioning at least two conductors on the probe at the distal end thereof;
providing at least two flexible conductive splines, each conductive spline having a first termination and a second termination;
electrically connecting the first terminations at a region on the probe axis beyond the distal end;
electrically connecting each second termination to a respective one of the conductors;
bending the splines into respective arcuate forms that encompass a volume; and
receiving voltages induced on the splines via the conductors and calculating a position and orientation of the volume in response to the received voltages.

The present invention will be more fully understood from the following detailed description of the embodiments thereof, taken together with the drawings in which:

### BRIEF DESCRIPTION OF THE DRAWINGS

FIG. 1 is a schematic illustration of a medical system comprising a medical probe, according to an embodiment of the present invention;

FIGS. 2A, 2B, and 2C are schematic illustrations of a distal end of the probe, according to an embodiment of the present invention;

FIG. 3 illustrates voltages developed between different terminations of conductors on the probe and a common termination of the conductors, according to an embodiment of the present invention;

FIG. 4 is a flowchart of steps of a balloon catheter algorithm implemented by a processor for a balloon, according to an embodiment of the present invention;

FIG. 5 illustrates experimental results, using a system similar to the medical system of FIG. 1, according to an embodiment of the present invention; and

FIG. 6 is a flowchart describing steps of a position and orientation algorithm for tracking a single axis sensor of a catheter, according to an embodiment of the present invention.

### DETAILED DESCRIPTION OF EMBODIMENTS

### Overview

It is important, for a basket or balloon catheter probe, that the location of the catheter is known as accurately as possible. Typically, this knowledge is acquired from one or more sensors that are incorporated into the catheter. In contrast, embodiments of the present invention use the splines of the basket or balloon catheter as individual single turn single axis magnetic sensors, the signals from which, when in an alternating magnetic field, providing a location for the sensors. A large area single turn sensor is as accurate as a multi-turn small area sensor because the overall areas of the two types of sensors are similar. The signals from the splines may be used on their own, or together with other location sensors incorporated in the catheter, to provide the location of a volume encompassed by the splines of the catheter.

Thus, an embodiment of the present invention comprises a probe that can be inserted into an organ, typically the heart, of a human patient. The probe, typically cylindrical, defines an axis of symmetry. At least two conductors are positioned on the probe at a distal end thereof, and the probe comprises at least two flexible conductive splines. Each conductive spline has a first termination and a second termination, and the first terminations are electrically connected together at a region on the probe axis beyond the probe distal end. Each second termination is electrically connected to a respective one of the conductors, and the splines are configured to bend in respective arcuate forms that encompass a volume.

A processor receives voltages induced on the splines via the conductors, the voltages being induced on the splines by an alternating magnetic field traversing the volume encompassed by the splines. The processor calculates a position and an orientation of the volume in response to the received voltages.

### System Description

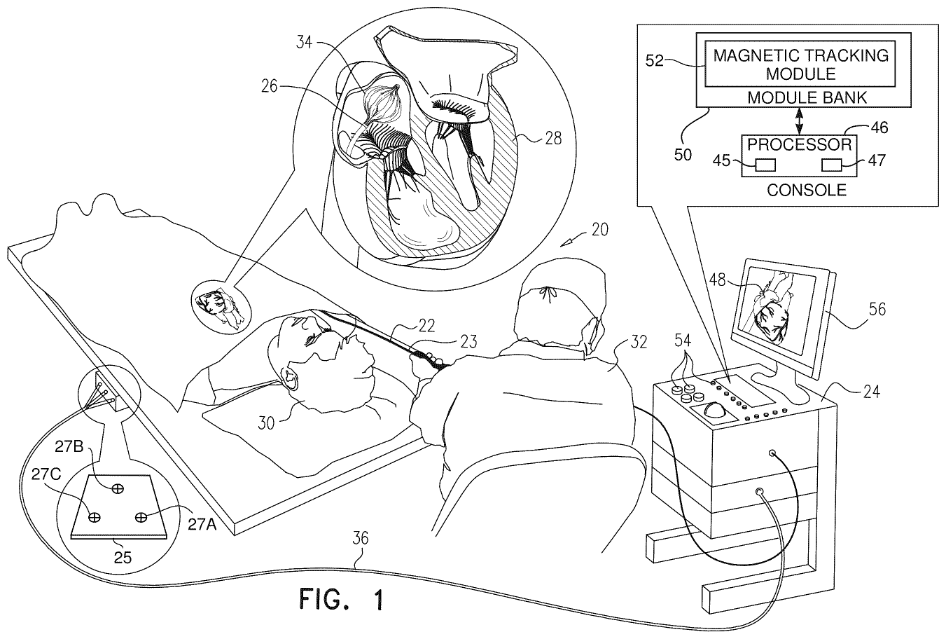

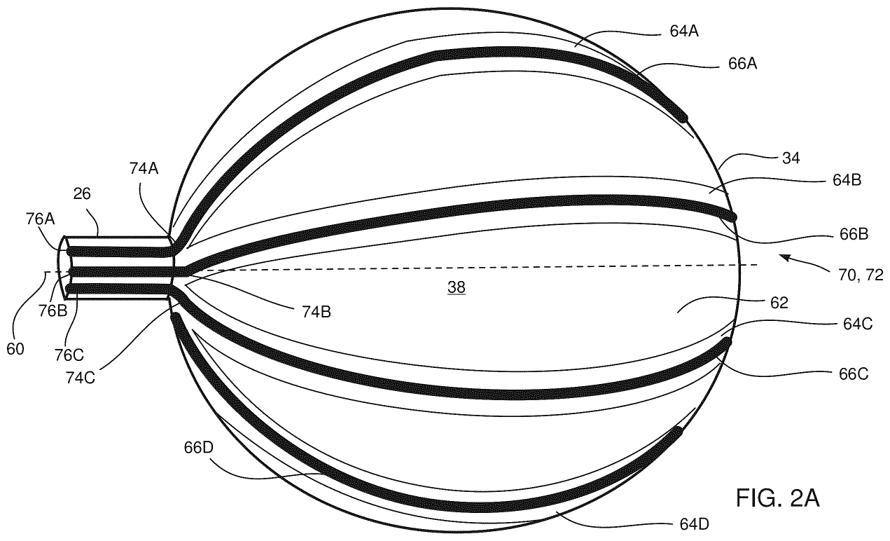

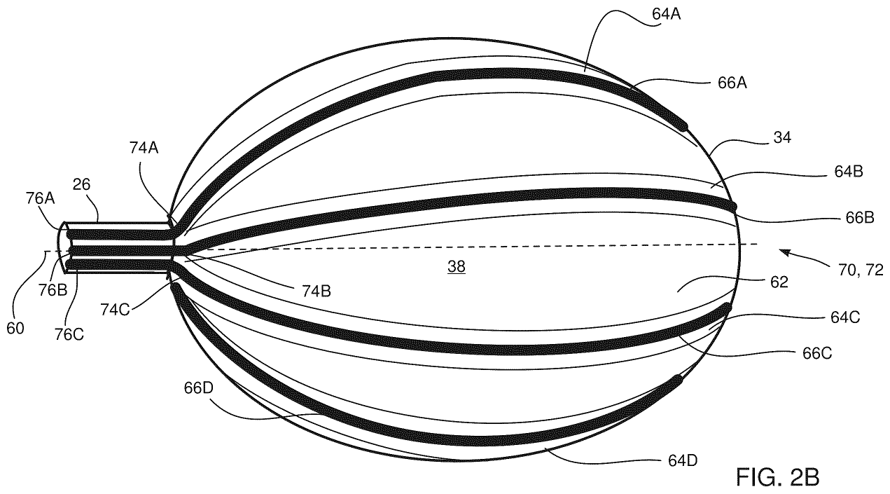

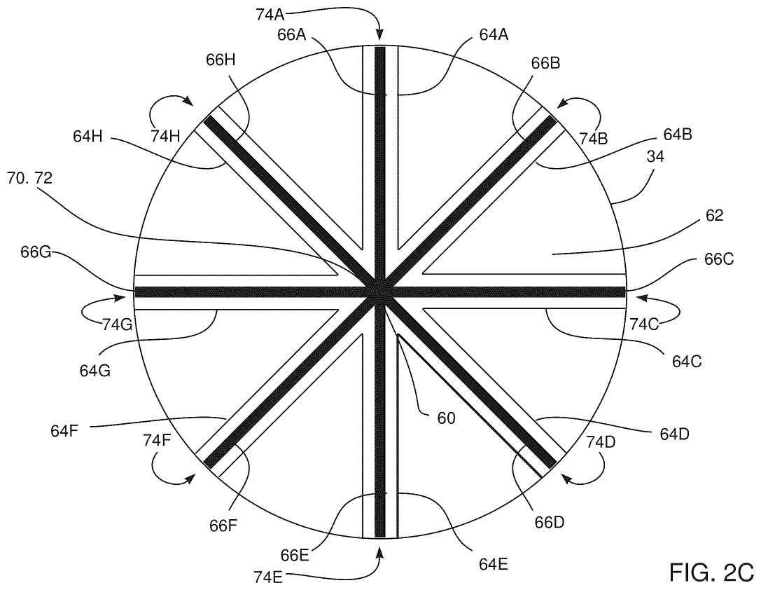

FIG. 1 is a schematic illustration of a medical system 20, comprising a medical probe 22 having a proximal end 23 and a distal end 26, according to an embodiment of the present invention. FIGS. 2A, 2B, and 2C are schematic illustrations of distal end 26 of probe 22, according to an embodiment of the present invention. System 20 may be based, for example, on the CARTO system produced by Biosense Webster Inc., of 33 Technology Drive, Irvine, CA 92618 USA.

In embodiments described hereinbelow, medical probe 22 is used for diagnostic or therapeutic treatment, such as for mapping electrical potentials and/or for performing ablation procedures in a heart 28 of a patient 30. Alternatively, probe 22 may be used, mutatis mutandis, for other therapeutic and/or diagnostic purposes in the heart or in other body organs.

During a medical procedure using system 20, a medical professional 32 inserts medical probe 22 into a biocompatible sheath (not shown) that has been pre-positioned in a lumen of the patient so that a balloon 34, described in more detail with reference to FIGS. 2A, 2B, and 2C, affixed to distal end 26 of the medical probe enters a chamber of heart 28.

System 20 is controlled by a system processor 46 which may be embodied as a single processor, or as a cooperatively networked or clustered set of processors. Processor 46 is typically a programmed digital computing device comprising a central processing unit (CPU), random access memory (RAM), non-volatile secondary storage, such as a hard drive or CD ROM drive, network interfaces, and/or peripheral devices. Program code, including software programs, and/or data are loaded into the RAM for execution and processing by the CPU and results are generated for display, output, transmittal, or storage, as is known in the art. Using its CPU and memories, processor 46 can be programmed to perform algorithms disclosed herein, using one or more modules, described hereinbelow, contained in a module bank 50 with which the processor communicates.

While for simplicity in the description herein processor 46 is assumed to be as described above, it will be understood that the scope of the invention includes a processor formed from any suitable integrated circuits, including, but not limited to, an ASIC (application specific integrated circuit), an FPGA (field-programmable gate array), an MCU (microcontroller unit), and a CPU.

In some embodiments processor 46 comprises real-time noise reduction circuitry 45, typically configured as an FPGA, followed by an analog-to-digital (A/D) signal conversion integrated circuit 47. The processor can pass the signals from A/D circuit 47 to modules described herein. The processor uses circuitry 45 and circuit 47, as well as features of the modules referred to above, in order to perform the algorithms.

Processor 46 is typically located in an operating console 24 of the system. Console 24 comprises controls 54 which are used by professional 32 to communicate with processor 46. Console 24 typically comprises a screen 56 upon which visual information generated by the processor, such as a map 48 of heart 28, may be presented to professional 32.

Console 24 is connected by a cable 36 to a location pad 25, typically situated beneath patient 30, comprising a plurality of fixed alternating magnetic field radiators. In one embodiment there are three sets of generally similar radiators 27A, 27B, and 27C, cach radiator comprising three orthogonal coils which radiate respective magnetic fields at different frequencies, so that in this case there are nine separate fields that are radiated. Radiators 27A, 27B, and 27C, collectively herein termed radiators 27, are powered by a magnetic tracking module 52 in module bank 50, and radiate their magnetic fields into a volume including heart 28 and its surroundings.

In addition to powering radiators 27, module 52 is configured to record voltages developed by conductive elements on balloon 34, the voltages being created in response to the alternating magnetic fields, generated by radiators 27, that traverse the conductive elements. The voltage generation is described in more detail below, and, as is also described below, from the recorded voltages, processor 46 is able to derive the position and orientation of balloon 34.

FIGS. 2A and 2B are respectively schematic perspective illustrations of balloon 34 in a generally spherical form and in a generally ellipsoidal form, and FIG. 2C is a schematic illustration of balloon 34 as viewed from a point distal to the balloon. Balloon 34 is assumed to enclose a volume 38. FIG. 2A illustrates the balloon when fully inflated, and FIG. 2B illustrates the balloon in an at least partially inflated configuration. Typically, when partially or completed inflated, balloon 34 has a diameter of the order of about 20 mm to about 40 mm.

As stated above, balloon 34 is fixed to distal end 26, and the distal end defines a probe axis of symmetry 60 of the balloon when it is at least partially inflated. In order to convey the balloon through the prepositioned sheath referred to above, the balloon is initially in a deflated form, and in this form distal end 26 is inserted into heart 28. Once in place in heart 28, the balloon may be inflated, typically by injecting a fluid, such as saline solution, into the balloon. Once the procedure for which the balloon has been positioned in heart 28 has been completed, the balloon may be deflated and probe 22 (with the deflated balloon) may be withdrawn from patient 30.

So that the balloon may be deflated and inflated, balloon 34 is formed from a biocompatible flexible plastic material 62, and the material is fixed to a plurality of generally similar flexible splines 64A, 64B, . . . 64H. Splines 64A, 64B, . . . are generically referred to herein as splines 64 and the splines are typically distributed symmetrically about axis 60. In the disclosure and in the claims, a spline is assumed to be a long, narrow, thin strip or slat. Furthermore, because of its shape, a spline may be bent into a generally arcuate form.

While the number of splines 64 may be any convenient odd or even number of splines that is two or more, in the following description, by way of example, there are assumed to be eight splines 64A, 64B, . . . 64H. In some embodiments splines 64 are internal to material 62, so that the splines act as ribs or spines covered by material 62. Alternatively, splines 64 are external to material 62, and the splines are attached by cement to the external surface of material 62 so as to support the material in place. Typically splines 64 are formed from flexible printed circuit (PC), a flexible wire such as nitinol, or a composition of such materials.

As is illustrated in FIGS. 2A and 2B, splines 64 surround, i.e., encompass, volume 38 enclosed by balloon 34.

For simplicity and clarity, in the following description, splines 64 are assumed to be external to material 62 and to be formed from flexible PC, and those having skill in the art will be able to adapt the description, mutatis mutandis, for the case of splines 64 being internal to the balloon material, and/or being formed from other materials referred to above.

Splines 64 typically comprise other elements, such as sensors, typically thermocouples or thermistors, to measure the temperature of heart tissue contacted by the splines, and electrodes. The electrodes may be used, inter alia, for radiofrequency (RF) ablation of the heart tissue, and/or for measuring and recording electrocardiogram (ECG) signals generated by the heart tissue. In some embodiments the other elements also comprise location sensors, typically coils, which provide signals in response to magnetic fields from radiators 27 traversing the sensors. Processor 46 may be configured to use such signals to find the location, i.e. the position and orientation, of the sensors. However, in some embodiments there are no such location sensors, since, as is described below, processor 46 uses signals from conductors in splines 64 to determine the position and the orientation of balloon 34.

Signals to and from such other elements are typically analyzed by, and/or generated by, processor 46 together with respective modules in module bank 50. For simplicity, such other elements and their respective modules are not shown in the figures.

Each spline 64A, 64B, . . . 64H comprises a respective conductor 66A, 66B, . . . 66H, the conductors being generically referred to herein as conductors 66. Conductors 66 may be formed on splines 64 by any convenient method, such as, but not limited to, by plating onto the splines.

Thus splines 64 are also referred to herein as conductive splines 64. Conductors 66 have a common first termination 70 at a distal region 72 of the balloon, region 72 being beyond distal end 26 and being on axis 60, where the axis cuts material 62. In addition, conductors 66A, 66B, . . . 66H have respective second, separated, terminations 74A, 74B, . . . 74H, collectively referred to as 74. Signals from terminations 74A, 74B, . . . 74H, produced as described below, are conveyed by respective conductors 76A, 76B, . . . 76H to proximal end 23 of probe 22 and then to module 52, and processor 46 uses the module to analyze the signals, as is also described below.

In some embodiments one or more of conductors 66 may be configured to perform multiple functions, such as being able to act as the electrodes, and/or as at least one terminal of the temperature sensors, and/or as at least one terminal of the location sensors, all of which are referred to above.

When balloon 34 is at least partially inflated, each pair of conductors 66 is connected at common termination 70, terminates at respective different second terminations 74, and encloses a region defined by the specific pair of conductors 66 (i.e., defined by 66A, 66B, 66C . . . 66H). It will be understood that the specific pair of conductors 66 acts as a coil having a single turn. Thus, when the region enclosed by the single turn coil is traversed by alternating magnetic fields from radiators 27, Faraday's law of induction provides that an induced voltage is developed across the different second terminations 74 of the pair 66, and that the voltage depends on the area of the region enclosed, the intensity of the magnetic fields at the region, and the orientation of the region with respect to the magnetic fields.

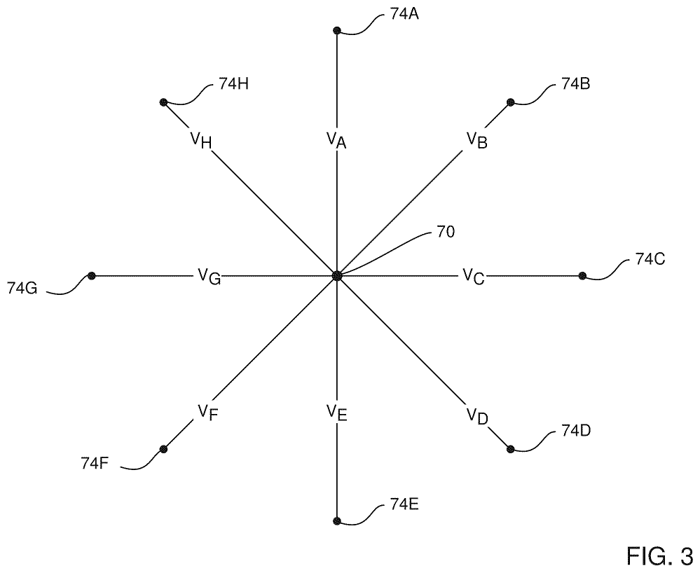

FIG. 3 illustrates the voltages developed between different terminations 74A, 74B, . . . 74H and common termination 70, according to an embodiment of the present invention. As is illustrated in the figure, voltages VA, VB, . . . . VH may be considered to be generated between second terminations 74A, 74B, . . . 74H and common first termination 70 of conductors 66, and the measured voltage between any two second terminations is the sum of the two voltages assumed to be generated on the two conductors. For example, a measured voltage VAC between terminations 74A and 74C is assumed to be given by equation (1):

$$\\ \begin{matrix}{V}_{AC} = {V}_{A} + {V}_{C} & ( 1 )\end{matrix}$$

Single axis sensors (SASs) having a coil with multiple turns are known in the art, and providing they are positioned in alternating magnetic fields that have been spatially mapped, it will be understood that the voltage developed across the coil can be used to find the position and orientation of the coil in the magnetic field. The Appendix below describes an algorithm for finding the position and orientation of an SAS in a mapped magnetic field, and those persons skilled in the art will be able to use the description of the algorithm, mutatis mutandis, to find the position and orientation of a single turn coil, such as a specific single turn coil defined by a pair of conductors 66. The algorithm is applicable since, inter alia, the overall area of a multiple turn SAS, typically having a diameter of the order of 1 mm, is of the same order as a single turn coil formed by a pair of conductors 66 on a balloon having a diameter of the order of 20 mm, so that the voltages formed by the multiple turn coil and the single turn coil (in the same magnetic field) are also of the same order.

For n conductors 66 (in splines 64), where n is an integer equal to or greater than 2, there are

$$\\ ( \begin{matrix}n \\ 2\end{matrix} )$$

different possible pairs of conductors forming single turn coils generating

$$\\ ( \begin{matrix}n \\ 2\end{matrix} )$$

respective voltages. Thus, for the 8 conductors (in their respective splines) considered here, there are 28 possible different single turn coils. This relationship would govern different single turn coils regardless of the number of n conductors. For example, where n=4, there are 6 possible different single turn coils; where n=6, there are 15 possible different single turn coils; where n=12, there are 66 possible different single turn coils and so on.

The voltage across each single turn coil gives the position and orientation of the coil, and the geometric relationships between the conductors, as well as the geometric relationships of the conductors to the balloon, are known or can be estimated. From the geometric relationships, and from the voltages developed by the 28 different single turn coils, processor 46 is able to estimate the position and orientation of volume 38 of balloon 34.

It will thus be understood that for n splines forming

$$\\ ( \begin{matrix}n \\ 2\end{matrix} )$$

pairs of single turn coils, from the geometric relationships, and from the voltages developed by the

$$\\ ( \begin{matrix}n \\ 2\end{matrix} )$$

coils, processor 46 is able to estimate the position and orientation of volume 38 of balloon 34.

Furthermore, rather than using all the

$$\\ ( \begin{matrix}n \\ 2\end{matrix} )$$

pairs of single turn coils, processor 46 may be configured to estimate the position and orientation of volume 38 of balloon 34 using a selected subset of the coils.

Thus, in a disclosed embodiment, rather than analyzing the 28 different voltages generated by the set of eight conductors 66, processor 46 is configured to analyze the four sets of voltages generated by the subset of the eight conductors comprising four opposing pairs of conductors 66, (66A, 66E), (66B, 66F), (66C, 66G), (66D, 66H). I.e., the processor records and analyzes the voltages given by equations (2):

$$\\ \begin{matrix}{V}_{AE} = {V}_{A} + {V}_{E} & ( 2 )\end{matrix}$$

$$\\ {V}_{BF} = {V}_{B} + {V}_{F}$$

$$\\ {V}_{CG} = {V}_{C} + {V}_{G}$$

$$\\ {V}_{DH} = {V}_{D} + {V}_{H}$$

Each opposing pair of conductors 66 (e.g., 66A and 66E forming one pair) in general forms a planar ellipse. (In the case of full inflation of balloon 34 the ellipse is approximately circular with an approximately unity ellipticity.) Furthermore, the centers of each of the four ellipses are approximately the same, corresponding to the center of volume 38. Because of the symmetry of splines 64 each of the four ellipses typically has substantially the same ellipticity, so that balloon 34 is effectively an ellipsoid of revolution around axis 60. Because, by virtue of the known construction of splines 64 on balloon 34, the orientation of the four ellipses with respect to each other is known, these orientations may be used to calculate an orientation of the balloon and a magnitude of its enclosed volume. By estimating the location of the balloon, i.e. its position and orientation, as well as its volume, the processor is able to provide a virtual representation of the actual size of the physical balloon and its actual location relative to the structures of the heart in a medical procedure.

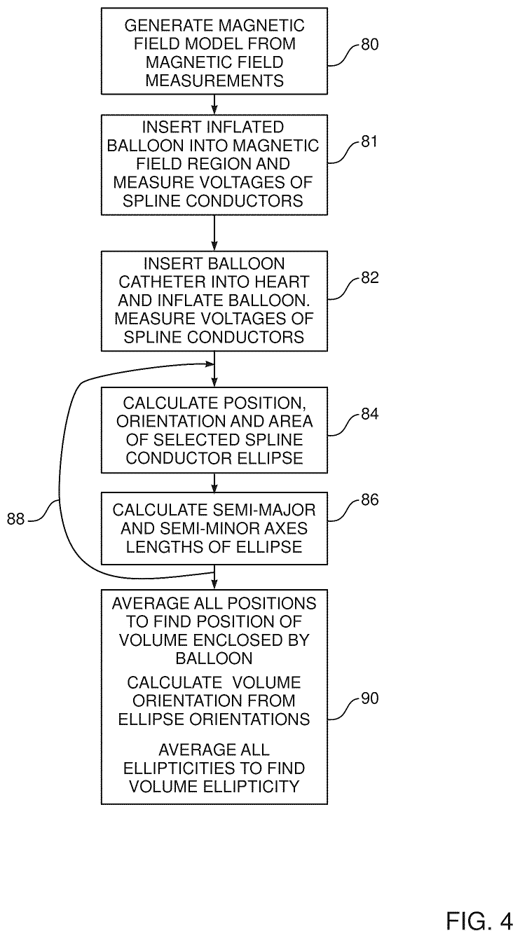

FIG. 4 is a flowchart of steps of a balloon catheter algorithm implemented by processor 46 for balloon 34, according to an embodiment of the present invention. The algorithm assumes that voltages of the four pairs of opposing conductors 66 of the disclosed embodiment described above, describing the four ellipses of the balloon, are measured. From the measured voltages, processor 46 finds the position and orientation of volume 38.

In a generation step 80, a magnetic field model \vec{B_{model}}(x, y, z) is generated from magnetic field measurements made in a region (that will surround heart 28 in a subsequent step of the algorithm) by a magnetic sampling detector scanned within the region at predefined points. The magnetic fields in the region are provided by the nine radiators 27, which are typically configured to transmit simultaneously at nine different frequencies. The magnetic field model, which provides a correspondence between a position (x,y,z) and the nine measured magnetic fields at the position, is fitted to the magnetic field measurements at the predefined points.

In a calibration step 81, balloon 34 is inflated, typically so that the balloon is approximately spherical, and the area of each of the four ellipses is measured. If the balloon is approximately spherical, then the ellipses are approximately circles, and the area of each ellipse is the same and is known from the balloon diameter. The area of each ellipse is herein assumed to be Area_{cal}.

The inflated balloon is inserted into a known magnetic field Φ, typically produced by a Helmholtz coil, and voltages Meas_{cal} of each of the four ellipses are measured and recorded by processor 46. It will be understood that there are nine voltages for each ellipse (from the nine radiators 27). From Faraday's law of induction the voltage is directly proportional to the product of Area_{cal} and a projection Φₚ of the magnetic field Φ onto the ellipse, so that

$$\\ \begin{matrix}{Meas}_{cal} = k {Area}_{cal} Φ p & ( 3 )\end{matrix}$$

where k is a constant of proportionality.

The processor stores the value of k for use in an ellipticity calculation step 86.

In a catheter insertion step 82, patient 30 is moved so that their heart is in the magnetic field region, and probe 22 is inserted into the patient so that balloon 34 enters the heart of the patient. The balloon is then inflated, and voltages Meas generated at second terminations 74A, 74B, . . . 74H, of spline conductors 66, are measured and recorded by processor 46 using module 52. For each ellipse formed by opposing conductors 66, there are nine different voltages generated by the nine magnetic fields from radiators 27.

In a location calculation step 84, processor 46 calculates the position and orientation of a specific ellipse, using the nine voltages measured and the magnetic field model \vec{B_{model}}(x, y, z) derived in step 80. Methods for calculating the position and orientation of a coil in a plurality of alternating magnetic fields are known in the art, and are used, for example, in the CARTO system referred to above. A method for calculating the position and orientation of a coil in a plurality of alternating magnetic fields is described in U.S. Pat. No. 8,818,486 to Montag, which is incorporated herein by reference. In addition, a method for calculating the position and orientation of a coil in a plurality of alternating magnetic fields is described in the Appendix below. The two latter methods are based on minimization. In step 84 the processor may also calculate the area of the specific ellipse, typically using a method as described in the Appendix below. Alternatively or additionally, the area may be calculated as described below in step 86.

In ellipticity calculation step 86, processor 46 calculates the ellipticity of the specific ellipse, i.e., the ratio between the semi-major axis length a and the semi-minor axis length b using magnitudes of the nine voltages Meas measured in step 82. Values of the magnitudes enable the processor to estimate an area A of the specific ellipse, as is explained below.

The ellipses of calibration step 81 are typically deformed in step 82, having an unknown area A. However, as stated above, the voltages produced by any given ellipse are directly proportional to the area of the ellipse and to the projection of the magnetic field onto the ellipse, so that using equation (3) the following equation holds:

$$\\ \begin{matrix}Meas = kA [ ( {v}_{x} , {v}_{y} , {v}_{z} ) \cdot {{B}_{model}}^{⟶} ( x , y , z ) ] & ( 4 )\end{matrix}$$

where (vₓ,V_{y},V_{z}) is a direction vector subject to the constraint that ∥vx, vy, vz∥=1.

In step 86 processor 46 uses equation (4), and the value of constant k derived in step 81, to estimate the area A of each ellipse. In equation (4) there are six unknowns; however, since there are nine independent radiators 27, there is more than sufficient information for processor 46 to use equation (4) to estimate a given ellipse area.

Equations (5) and (6) below are equations respectively relating the area A of an ellipse to a and b, and a perimeter p of the ellipse to a and b. The value of perimeter p is the total length of the conductors 66 forming the ellipse, and this value is known.

$$\\ \begin{matrix}A = π ab & ( 5 )\end{matrix}$$

$$\\ \begin{matrix}p \approx π [ 3 ( a + b ) - \sqrt{( 3 a + b ) ( a + 3 b )} ] & ( 6 )\end{matrix}$$

Using the values of A and p, processor 46 solves equations (5) and (6) for a and b, and thus the ellipticity of the specific ellipse.

As indicated by an arrow 88, the processor repeats the calculations of steps 84 and 86 for all the four ellipses generated by opposing conductors 66.

In a concluding step 90 of the algorithm, the processor averages the four positions of the ellipses to find the position of volume 38. The processor also finds an orientation of volume 38 from the four ellipse orientations. An average of the ellipticities of the ellipses gives a value for the ellipticity of the ellipsoid of revolution of volume 38.

Since volume 38 is an ellipsoid of revolution, a magnitude V of this volume, i.e. the volume enclosed by balloon 34, is given by equation (7):

$$\\ \begin{matrix}V = \frac{4 π}{3} {ab}^{{}_{} 2} & ( 7 )\end{matrix}$$

In a disclosed embodiment, using the position, orientation, and magnitude of volume 38 as determined above, the processor may present in step 90, on map 48 of the heart (FIG. 1), a virtual representation of the actual size of balloon 34 and the balloon's actual location relative to the structures of the heart. The presentation may be implemented during a medical procedure.

Alternatively or additionally, the processor may present on screen 56 a numerical value of magnitude V. Professional 32 may estimate from the numerical value, and/or from the virtual representation, if balloon 34 has been underinflated or overinflated.

While the description above for the flowchart of FIG. 4 considers splines forming four pairs of coils, those persons skilled in the art will be able to adapt the description, mutatis mutandis, for other possible numbers of pairs of coils, and all such possible numbers are considered to be within the scope of the present invention.

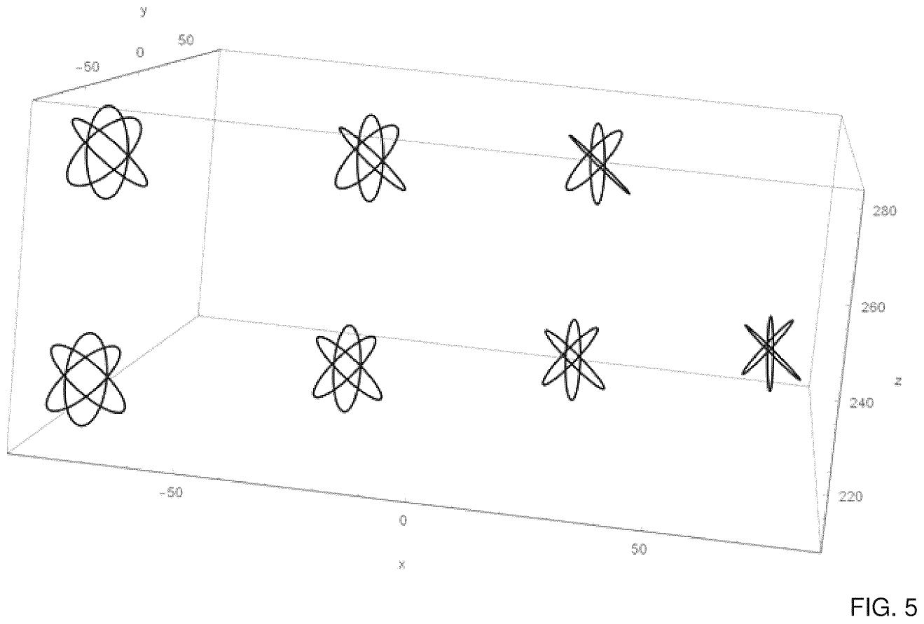

FIG. 5 illustrates experimental results obtained by the inventors, using a system similar to system 20, according to an embodiment of the present invention. The results are for the four ellipse systems described above with reference to FIGS. 3 and 4. The figure shows, for seven selected positions of volume 38 enclosed by balloon 34, three of the four ellipses that processor 46 calculates at each of the seven positions.

### Appendix

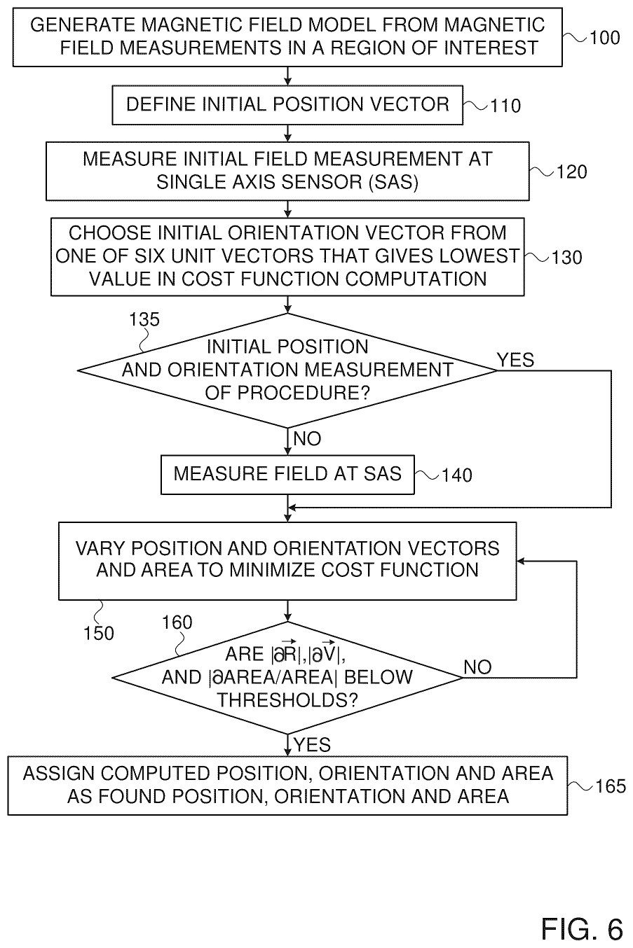

FIG. 6 is a flowchart describing steps of a position and orientation (P&O) algorithm for tracking a single axis sensor (SAS) of a catheter, according to an embodiment of the present invention. The flowchart assumes that the SAS comprises one or more turns of a coil, and that the SAS is in a region of interest (ROI) irradiated by i alternating magnetic fields generated by i respective magnetic field generators, where i is a positive integer. In the following description i=9, corresponding to radiators 27 described above, and the ROI may be assumed to comprise a region containing heart 28. The P&O algorithm is assumed to be implemented by processor 46 using magnetic tracking module 52.

In a generation step 100, a magnetic field model \vec{B_{model}}(x, y, z), substantially the same as step 80 of the balloon catheter algorithm, is generated from magnetic field measurements made in the ROI by a magnetic sampling detector scanned within the ROI at predefined points. The magnetic field model is fitted to the magnetic field measurements at the predefined points.

At a definition step 110, an initial position vector is defined by arbitrarily assigning an initial SAS position vector \vec{r}=(x, y, z) of the distal tip of the catheter, at a point such as a center of the ROI.

In a first measurement step 120, i initial magnetic field measurements, measᵢ, are measured at the single axis sensor. The field measurements are received by module 52 and relayed to processor 46.

In a choosing step 130, an initial orientation vector is chosen from one of six unit vectors, e.g., ((1,0,0), (−1,0,0), (0,1,0), (0,−1,0), (0,0,1), (0,0,1)). Processor 46 computes six cost functions cost using equations (A)-(C) below:

$$\\ \begin{matrix}Δ {Meas}_{i} = {Mest}_{i} - {meas}_{i} & ( A )\end{matrix}$$

$$\\ \begin{matrix}cost = {\sum }_{i = 1}^{10} Δ {Meas}_{i}^{{}_{} 2} & ( B )\end{matrix}$$

where ΔMeas₁₀ is a penalty function given by

$$\\ \begin{matrix}Δ {Meas}_{10} = constraintWeight ( {v}_{x}^{{}_{} 2} + {v}_{y}^{{}_{} 2} + {v}_{z}^{{}_{} 2} - 1 ) & ( C )\end{matrix}$$

vₓ, v_{y}, v_{z}, are components of an SAS orientation vector \vec{v}=(vₓ, v_{y}, v_{z}),

$$\\ {Mest}_{i} = \frac{Area}{{Area}_{cal}} ( {v}_{x} , {v}_{y} , {v}_{z} ) \cdot {{B}_{model} ( x , y , z )}_{i}$$

Area is an actual area of the SAS,
Area_{cal} is the calibrated area of the SAS,
and constraintWeight is a constant typically with a value of constraintWeight=0.05.

In step 130 processor 46 uses the initial field measurement from step 120, the six unit vectors, and the initial position vector defined in step 110. The initial orientation vector chosen is the one that gives the lowest value of cost in six cost function computations of equation (B).

In a first decision step 135, if this is the initial position and orientation measurement, processor 46 bypasses a second measurement step 140, since the fields were already measured in step 120. If this is not the initial measurement, fields measᵢ are measured at the single axis sensor in second measurement step 140.

In a varying step 150 and a second decision step 160, processor 46 initiates an iteration loop after step 140 to minimize the cost function value cost. In step 150 SAS position vector \vec{r}=(x, y, z) and SAS orientation vector \vec{v}=(vₓ, v_{y}, v_{z}) are varied to reduce the cost function.

While in the iteration loop, not only is the cost function reduced, typically monotonically, but processor 46 also computes the seven differential variables (∂x, ∂y, ∂z, ∂vₓ, ∂v_{y}, ∂v_{z}, ∂Area) using a Levenberg-Marquardt (L-M) variation of the Gauss-Newton (G-N) optimization method, according to equation (D):

$$\\ \begin{matrix}( \begin{matrix}\partial x \\ \partial y \\ \partial z \\ \partial {v}_{x} \\ \partial {v}_{y} \\ \partial {v}_{z} \\ \partial Area\end{matrix} ) = - {( {J}^{{}_{} T} J + λ diag ( J ) )}^{- 1} {J}^{{}_{} T} Δ Meas & ( D )\end{matrix}$$

where J is the Jacobian matrix, J^{T} is the transpose of J, diag(J) is a diagonal matrix whose elements are the diagonal elements of J, and λ is a non-negative scalar parameter, which is typically approximately 0.001, and which shrinks by a factor of 10 in every iteration. ΔMeas in equation (D) is a 10×1 matrix, which comprises nine terms from the nine radiators 27 and one penalty function term from equation (C).

The seven differentials (∂x, ∂y, ∂z, ∂vₓ, ∂v_{y}, ∂v_{z}, ∂Area) computed iteratively from equation (D) represent the differential changes of the components of position vector \vec{r}=(x, y, z), the differential changes of the components of orientation vector \vec{v}=(vₓ, v_{y}, v_{z}), and the change in area between successive iteration loop cycles.

Using the seven differentials, processor 46 calculates a change in the position vector |∂\vec{r}|=\sqrt{(∂x)²+(∂y)²+(∂z)²}, a change in the orientation vector |∂\vec{v}|=\sqrt{(∂vₓ)²+(∂v_{y})²+(∂v_{z})²}, and a change in the area ∂Area between iteration loop cycles.

In a second decision step 160, when |∂\vec{r}|, |∂\vec{v}|, and |∂Area|_{A̅r̅e̅a̅} are not below a predefined threshold, typically 0.002, the iteration loop continues with varying step 150. If |∂\vec{r}|, |∂\vec{v}|, and |∂Area|_̅{̅A̅r̅e̅a̅}̅ are below the predefined threshold, processor 46 assigns the computed position, orientation, and area as the found position, orientation, and area in an assignment step 165, i.e. the measured position and orientation vector, and the area, of the SAS.

While the description above has assumed splines forming a balloon catheter positioned in a heart, it will be understood that embodiments of the present invention may be implemented for splines forming a basket catheter. It will also be understood that embodiments of the present invention may be used for procedures on organs other than the heart, such as sinuplasty procedures.

It will thus be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art.

1. A medical probe, comprising:
an elongated shaft defining a longitudinal probe axis;
a flexible spline assembly affixed to a distal end of the elongated shaft, the flexible spline assembly comprising a plurality of spline conductors electrically coupled to each other at a common termination at a distal portion of the flexible spline assembly, each spline conductor of the plurality of spline conductors extending in an arcuate shape to a proximal portion of the flexible spline assembly; and
a plurality of elongated conductors each electrically coupled to a respective spline conductor of the plurality of spline conductors and extending through the elongated shaft to a proximal end of the elongated shaft.

2. The medical probe of claim 1, wherein the flexible spline assembly is configured to function as a location sensor in a volume with predetermined alternating magnetic fields.

3. The medical probe of claim 1, wherein each of the plurality of spline conductors are electrically isolated from each other but for the common termination.

4. The medical probe of claim 1,
wherein each of the plurality of spline conductors comprises a respective termination at the proximal portion of the flexible spline assembly, and
wherein each of the plurality of elongated conductors are electrically coupled to the respective termination of the respective spline conductor.

5. The medical probe of claim 1, wherein the plurality of spline conductors is configured to define an ellipsoidal volume comprising a diameter measuring approximately 20 millimeters.

6. The medical probe of claim 1, wherein the flexible spline assembly lacks a multi-turn inductive coil.

7. The medical probe of claim 1, further comprising:
a balloon affixed to the plurality of spline conductors.

8. The medical probe of claim 1, wherein the flexible spline assembly comprises a flexible printed circuit.

9. The medical probe of claim 1, wherein the flexible spline assembly comprises a temperature sensor.

10. The medical probe of claim 1, wherein the flexible spline assembly comprises an electrode.

11. A system comprising:
at least one processor; and
non-transitory computer-readable medium with instructions thereon, that when executed by the at least one processor, cause the system to:
provide an output to a plurality of radiators to thereby generate alternating magnetic fields,
measure a plurality of electrical signals across a plurality of pairs of a plurality of conductors electrically coupled to a distal portion of a medical probe such that the plurality of conductors encompass an ellipsoid volume and such that plurality of electrical signals is based in part on the alternating magnetic fields through the ellipsoid volume, and
calculate a position and an orientation of the ellipsoid volume based at least in part on the plurality of electrical signals.

12. The system of claim 11, wherein the non-transitory computer-readable medium comprises instructions thereon, that when executed by the at least one processor, cause the system to:
calculate a position and an orientation of the distal portion of the medical probe based at least in part on a geometric relationship of the plurality of conductors.

13. The system of claim 11, wherein the non-transitory computer-readable medium comprises instructions thereon, that when executed by the at least one processor, cause the system to:
calculate a position and an orientation of the distal portion of the medical probe without relying on an electrical signal from a multi-turn inductive coil.

14. The system of claim 11, wherein the ellipsoid volume has a diameter measuring approximately 20 millimeters.

15. The system of claim 11,
wherein the plurality of conductors comprises n conductors electrically coupled to the distal portion of a medical probe,
wherein the plurality of pairs comprises
$$\\ ( \begin{matrix}n \\ 2\end{matrix} )$$
pairs, and
wherein the non-transitory computer-readable medium comprises instructions thereon, that when executed by the at least one processor, cause the system to measure the plurality of electrical signals across the
$$\\ ( \begin{matrix}n \\ 2\end{matrix} )$$
pairs of the n conductors.

16. The system of claim 15, wherein n is equal to four, five, six, seven, eight, nine, ten, eleven, or twelve.

17. The system of claim 11, wherein at least one pair of the plurality of pairs defines an enclosed non-planar, arcuate surface area.

18. The system of claim 11,
wherein the plurality of conductors comprises n conductors electrically coupled to the distal portion of a medical probe,
wherein the plurality of pairs comprises n/2 pairs, and
wherein the non-transitory computer-readable medium comprises instructions thereon, that when executed by the at least one processor, cause the system to measure the plurality of electrical signals across the n/2 pairs of the n conductors.

19. The system of claim 18,
wherein each pair of the n/2 pairs of n conductors is configured to define an enclosed planar surface area, and
wherein the plurality of electrical signals is based in part on a portion of the alternating magnetic fields through the enclosed planar surface area causing induction in each pair of the n/2 pairs of n conductors.

20. The system of claim 18, wherein the enclosed planar surface area for each pair of the n/2 pairs of n conductors is approximately elliptical.
